(19) United States
(12) Patent Application Publication
(10) Pub. No.: US 2025/0099631 A1
(43) Pub. Date: Mar. 27, 2025

(54) COMPLEXES FOR CANCER TREATMENT AND IMAGING

(71) Applicant: SCIENCONS AS, OSLO (NO)

(72) Inventor: Roy H. LARSEN, Oslo (NO)

(21) Appl. No.: 18/729,758

(22) PCT Filed: Jan. 20, 2023
(86) PCT No.: PCT/EP2023/051355

§ 371 Date: Jul. 17, 2024

(30) Foreign Application Priority Data
Jan. 21, 2022 (EP) 22152725.2

(51) Int. Cl. A61K 51/04; A61P 35/04

(52) U.S. Cl. CPC A61K 51/0482; A61P 35/04

(57) ABSTRACT

The present invention relates to complexes comprising a PSMA targeting compound linked to a radionuclide such as ⁴⁷Sc, ¹¹¹In, ¹⁶¹Tb. These compounds, and pharmaceutical compositions comprising them, can be used for medical applications, including in the treatment of cancer as well as in monitoring and diagnostics.

### FIELD

The present invention relates to complexes comprising a PSMA targeting compound linked to a radionuclide such as ¹¹¹In, ¹⁶¹Tb, ⁴⁷Sc, ⁶⁸Ga, ⁴⁴Sc. These compounds, and pharmaceutical compositions comprising them, can be used for medical applications, including in the treatment of cancer as well as in monitoring and diagnostics.

### BACKGROUND

Prostate cancer is among the most frequent causes of cancer related mortality in men. There is a great demand for new and effective treatment, especially in hormone refractory late stage disease. Skeletal metastases are a frequent problem in late stage disease and therefore the alpha-particle emitter ²²³Ra (Xofigo) was introduced as a bone specific therapy for late stage prostate cancer patients with skeletal metastases.

Although, as a bone-seeker, ²²³Ra shows significant clinical benefit for patients its activity is limited to the bone metastases and is not targeting soft tissue metastases.

Several carrier molecules for radioligand targeting of prostate specific membrane antigen (PSMA) exists. Lutetium-177 labeled PSMA-617 (¹⁷⁷Lu-PSMA-617) is the compound in most advanced clinical development stage for use in radionuclide therapy.

This molecule works in a suitable manner and give relevant tumor to normal tissue ratios for longer lived (i.e. a half-life of a few days) radionuclides, including ¹⁷⁷Lu and ²²⁵Ac, but at early times points (typically a few hours after injection) shows high uptake in kidneys. With shorter lived radionuclides like ²¹²Pb (half-life of 10.6 hours), the initial kidney uptake represents a potential toxicity problem.

It is therefore advantageous to use a PSMA-ligand with less kidney uptake, but this should not compromise the tumor uptake. Alternatively, a PSMA targeting radioligand with high initial kidney uptake can be combined with a radinuclide with longer half-life if the tumor retention is much stronger than the kidney retention. Also, a molecule with both high tumor and kidney retention at early time points can be used for diagnostic scanning, e.g., PET and SPECT to visualize tumor distribution and PSMA expression etc. The PSMA ligand molecules are made up of (1) a PSMA-binding region, (2) a linker region and (3) a chelator, whereby the linker region connects the (1) and (3). The linker region also is used to adjust molecular size and polarity etc to affect the in vivo distribution properties. The PSMA-binding region (motif) used in PSMA-617 is a structure that can be found in several molecules of this class, developed by several different inventors and researchers, including PSMA-11 and PSMA I&T as well as ¹³¹I and ²¹¹At labelled PSMA binding ligands.

New compounds that contain a PSMA region are warranted because currently all ligands in testing have challenges, including a relatively low radiobiological effectiveness (RBE) and suboptimal biodistribution.

There is also a need for an improved alpha emitter, beta emitter, Auger emitter, positron emitter and/or photon emitter that can target both the bone metastases and the soft tissue metastases, and for compounds that can be used in imaging and diagnostics.

The present invention relates to compounds and compositions that address these challenges.

### SUMMARY

An object of the present invention relates to a complex comprising a compound according to of the formula:

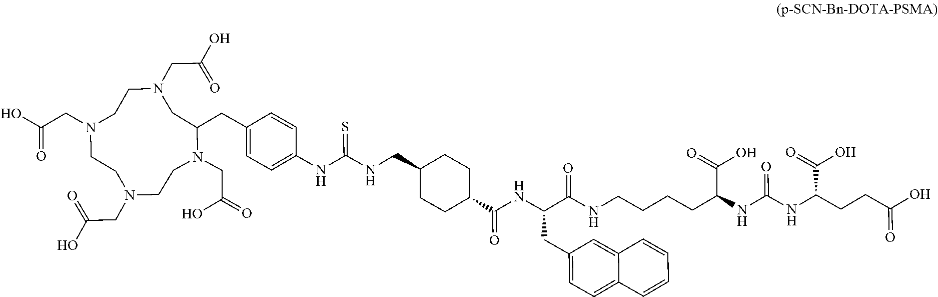 [SMILES: O=C(O)CC[C@H](NC(=O)N[C@@H](CCCCNC(=O)[C@H](Cc1ccc2ccccc2c1)NC(=O)[C@H]1CC[C@H](CNC(=S)Nc2ccc(CC3CN(CC(=O)O)CCN(CC(=O)O)CCN(CC(=O)O)CCN3CC(=O)O)cc2)CC1)C(=O)O)C(=O)O]

and b) a radionuclide selected from the group consisting of ²²⁷Th, ¹⁷⁷Lu, ²¹²Pb, ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ¹⁷⁷Lu, ²²⁵Ac, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb.

One or more embodiments of the present invention relates to the complex of the present invention, wherein the radionuclide is selected from the group consisting of ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb. One or more embodiments of the present invention relates to the complex of the present invention, wherein the radionuclide is selected from the group consisting of ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ¹⁷⁷Lu, ²²⁵Ac, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb.

One or more embodiments of the present invention relates to the complex of the present invention, wherein the radionuclide is ¹⁷⁷Lu. One or more embodiments of the present invention relates to the complex of the present invention, wherein the radionuclide is ²²⁵Ac. One or more embodiments of the present invention relates to the complex of the present invention, wherein the radionuclide is ¹¹¹In. One or more embodiments of the present invention relates to the complex of the present invention, wherein the radionuclide is ⁸⁶Y. One or more embodiments of the present invention relates to the complex of the present invention, wherein the radionuclide is ⁹⁰Y. One or more embodiments of the present invention relates to the complex of the present invention, wherein the radionuclide is ⁸⁹Zr. One or more embodiments of the present invention relates to the complex of the present invention, wherein the radionuclide is ⁶⁸Ga. One or more embodiments of the present invention relates to the complex of the present invention, wherein the radionuclide is ⁶⁴Cu. One or more embodiments of the present invention relates to the complex of the present invention, wherein the radionuclide is ¹⁶¹Tb. One or more embodiments of the present invention relates to the complex of the present invention, wherein the radionuclide is ⁶⁷Cu. One or more embodiments of the present invention relates to the complex of the present invention, wherein the radionuclide is ⁴⁴Sc. One or more embodiments of the present invention relates to the complex of the present invention, wherein the radionuclide is ⁴⁶Sc. One or more embodiments of the present invention relates to the complex of the present invention, wherein the radionuclide is ⁴⁷Sc. One or more embodiments of the present invention relates to the complex of the present invention, wherein the radionuclide is ⁴⁸Sc. One or more embodiments of the present invention relates to the complex of the present invention, wherein the radionuclide is ¹⁵⁵Tb. One or more embodiments of the present invention relates to the complex of the present invention, wherein the radionuclide is ¹⁴⁹Tb. One or more embodiments of the present invention relates to the complex of the present invention, wherein the radionuclide is ²⁰³Pb. One or more embodiments of the present invention relates to the complex of the present invention, wherein the radionuclide is ⁴³Sc. One or more embodiments of the present invention relates to the complex of the present invention, wherein the radionuclide is ¹⁶¹Tb. One or more embodiments of the present invention relates to the complex of the present invention, wherein the radionuclide is ¹⁷⁷Lu. One or more embodiments of the present invention relates to the complex of the present invention, wherein the radionuclide is ²²⁷Th. One or more embodiments of the present invention relates to the complex of the present invention, wherein the radionuclide is ²¹²Pb.

One or more embodiments of the present invention relates to the complex or the pharmaceutical composition of the present invention, for use in imaging.

One or more embodiments of the present invention relates to the complex or the pharmaceutical composition of the present invention, for use in positron emission tomography (PET) imaging or single-photon emission computerized tomography (SPECT) imaging.

One or more embodiments of the present invention relates to the use of the complex or the pharmaceutical composition of the present invention, wherein the imaging is for providing diagnosis, staging, and/or monitoring treatment of cancer.

One or more embodiments of the present invention relates to a method of treatment of malignant or non-malignant disease by administration of a pharmaceutical composition of the present invention to an individual in need thereof.

One or more embodiments of the present invention relates to a method of amelioration of malignant or non-malignant disease by administration of a pharmaceutical composition of the present invention to an individual in need thereof.

One or more embodiments of the present invention relates to a method of inhibition of malignant or non-malignant disease by administration of a pharmaceutical composition according of the present invention to an individual in need thereof.

One or more embodiments of the present invention relates to a kit comprising: a first vial comprising a pharmaceutical composition of the present invention, and a second vial comprising a neutralizing composition to adjust pH and/or isotonicity of the radiopharmaceutical composition prior to administration to a patient.

One or more embodiments of the present invention relates to a kit comprising: a first vial comprising a pharmaceutical composition comprising ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb; and a second vial comprising p-SCN-Bn-DOTA-PSMA.

### DETAILED DESCRIPTION

### Some Abbreviations Used

Peptide mimetic—also termed peptidomimetic, is a small protein-like chain designed to mimic a peptide. They typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. This can have a role in the development of drug-like compounds from existing peptides. These modifications involve changes to the peptide that will not occur naturally (such as altered backbones and the incorporation of nonnatural amino acids). Based on their similarity with the precursor peptide, peptidomimetics can be grouped into four classes (A-D) where A features the most and D the least similarities. Classes A and B involve peptide-like scaffolds, while classes C and D include small molecules.

PSMA—Prostate-specific membrane antigen. Synonyms PSMA, Prostate Specific Cancer Antigen, PSM, FGCP, FOLH, GCP2, mGCP, GCPII, NAALAD1, NAALAdase, FOLH1, Glutamate carboxypeptidase 2, Glutamate carboxypeptidase II, Membrane glutamate carboxypeptidase, N-acetylated-alpha-linked acidic dipeptidase I, Pteroylpoly-gamma-glutamate carboxypeptidase, Folylpoly-gamma-glutamate carboxypeptidase, Folate hydrolase 1, Prostate-specific membrane antigen, Cell growth-inhibiting protein 27

p-SCN-Bn-DOTA—2-(4-isothiocyanatobenzyl)-1,4,7,10-tetraazacyclododecane-1,4,7,10-tetraacetic acid

DOTA-1,4,7,10-tetraazacyclododecane-1,4,7,10-tetraacetic acid and also used for benzyl-1,4,7,10-tetraazacyclododecane-1,4,7,10-tetraacetic acid (e.g. conjugated to monoclonal antibody)

The invention is in the field of radiolabeled therapy and diagnostics agents, which are PSMA peptidomimetic agents. According to the invention, radiolabelled derivatives of urea-based prostate-specific membrane antigen (PSMA) inhibitors are disclosed.

Thus, an object of the present invention relates to a complex comprising a compound according to the formula:

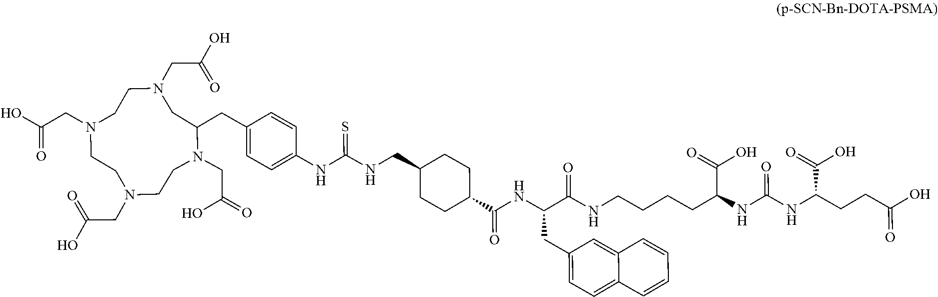 [SMILES: O=C(O)CC[C@H](NC(=O)N[C@@H](CCCCNC(=O)[C@H](Cc1ccc2ccccc2c1)NC(=O)[C@H]1CC[C@H](CNC(=S)Nc2ccc(CC3CN(CC(=O)O)CCN(CC(=O)O)CCN(CC(=O)O)CCN3CC(=O)O)cc2)CC1)C(=O)O)C(=O)O]

and b) a radionuclide selected from the group consisting of ²²⁷Th, ¹⁷⁷Lu, ²¹²Pb, ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ¹⁷⁷Lu, ²²⁵Ac, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb.

The radionuclides can also be selected from the group consisting of ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ¹⁷⁷Lu, ²²⁵Ac, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb.

A complex of p-SCN-Bn-DOTA-PSMA with ²⁰³Pb could therefore be written herein as ²⁰³Pb-p-SCN-Bn-DOTA-PSMA.

### Radionuclides

One or more embodiments of the present invention relates to the complex of the present invention, wherein the radionuclide is selected from the group consisting of ²²⁷Th, ¹⁷⁷Lu, ²¹²Pb, ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb.

The radionuclides can also be selected from the group consisting of ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ¹⁷⁷Lu, ²²⁵Ac, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb. The radionuclides can also be selected from the group consisting of ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb. One or more embodiments of the present invention relates to the complex of the present invention, wherein the radionuclide is ¹⁷⁷Lu. One or more embodiments of the present invention relates to the complex of the present invention, wherein the radionuclide is ²²⁵Ac. One or more embodiments of the present invention relates to the complex of the present invention, wherein the radionuclide is ¹¹¹In. One or more embodiments of the present invention relates to the complex of the present invention, wherein the radionuclide is ⁸⁶Y. One or more embodiments of the present invention relates to the complex of the present invention, wherein the radionuclide is ⁹⁰Y. One or more embodiments of the present invention relates to the complex of the present invention, wherein the radionuclide is ⁸⁹Zr. One or more embodiments of the present invention relates to the complex of the present invention, wherein the radionuclide is ⁶⁸Ga. One or more embodiments of the present invention relates to the complex of the present invention, wherein the radionuclide is ⁶⁴Cu. One or more embodiments of the present invention relates to the complex of the present invention, wherein the radionuclide is ¹⁶¹Tb. One or more embodiments of the present invention relates to the complex of the present invention, wherein the radionuclide is ⁶⁷Cu. One or more embodiments of the present invention relates to the complex of the present invention, wherein the radionuclide is ⁴⁴Sc. One or more embodiments of the present invention relates to the complex of the present invention, wherein the radionuclide is ⁴⁶Sc. One or more embodiments of the present invention relates to the complex of the present invention, wherein the radionuclide is ⁴⁷Sc. One or more embodiments of the present invention relates to the complex of the present invention, wherein the radionuclide is ⁴⁸Sc. One or more embodiments of the present invention relates to the complex of the present invention, wherein the radionuclide is ¹⁵⁵Tb. One or more embodiments of the present invention relates to the complex of the present invention, wherein the radionuclide is ¹⁴⁹Tb. One or more embodiments of the present invention relates to the complex of the present invention, wherein the radionuclide is ²⁰³Pb. One or more embodiments of the present invention relates to the complex of the present invention, wherein the radionuclide is ⁴³Sc. One or more embodiments of the present invention relates to the complex of the present invention, wherein the radionuclide is ¹⁶¹Tb. One or more embodiments of the present invention relates to the complex of the present invention, wherein the radionuclide is ¹⁷⁷Lu. One or more embodiments of the present invention relates to the complex of the present invention, wherein the radionuclide is ²²⁷Th. One or more embodiments of the present invention relates to the complex of the present invention, wherein the radionuclide is ²¹²Pb.

### A Pharmaceutical Composition

The compounds or complexes of the present disclosure are usually applied in the treatment or monitoring of diseases and are usually formulated in pharmaceutical compositions.

Such compositions are optimized for parameters such as physiological tolerance and shelf-life.

In one or more embodiment(s) of present disclosure relates to a pharmaceutical composition comprising a compound or complex of the present disclosure, and a pharmaceutically acceptable carrier and/or excipient.

In one or more embodiment(s) of present disclosure relates to a pharmaceutical composition comprising a compound or complex of the present disclosure, and a pharmaceutically acceptable and a diluent, carrier, surfactant, and/or excipient.

Acceptable pharmaceutical carriers include but are not limited to non-toxic buffers, fillers, isotonic solutions, etc. More specifically, the pharmaceutical carrier can be but are not limited to normal saline (0.9%), half-normal saline, Ringer's lactate, 5% Dextrose, 3.3% Dextrose/0.3% Saline. The physiologically acceptable carrier can contain a radiolytic stabilizer, e.g., ascorbic acid, which protect the integrity of the pharmaceutical during storage and shipment.

One or more embodiments of the present invention relates to a pharmaceutical composition of the present invention, which is dosaged with a radioactivity of 100 kBq to 50 GBq per dose.

### Imaging

One or more embodiments of the present invention relates to the use of the complex or the pharmaceutical composition of the present invention, wherein the imaging is for providing diagnosis, staging, and/or monitoring treatment of cancer. The imaging can be for providing diagnosis of cancer. The imaging can be for providing staging of cancer. The imaging can be for providing monitoring treatment of cancer.

An aspect of the present disclosure relates to a compounds, complexes and pharmaceutical compositions according to the present disclosure, wherein the radionuclide is suitable for imaging, i.e. can for example be selected from the group consisting of ²²⁷Th, ¹⁷⁷Lu, ²¹²Pb, ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ⁴³Sc, ⁴⁴Sc, ⁴⁶Sc, ⁴⁷Sc, ⁴⁸Sc, ¹⁵⁵Tb, ¹⁴⁹Tb, ¹⁶¹Tb, and ¹¹C, ¹³N, ¹⁵O, ¹⁸F. The radionuclides can also be selected from the group consisting of ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ⁴³Sc, ⁴⁴Sc, ⁴⁶Sc, ⁴⁷Sc, ⁴⁸Sc, ¹⁵⁵Tb, ¹⁴⁹Tb, ¹⁶¹Tb, and ¹¹C, ¹³N, ¹⁵O, ¹⁸F.

One or more embodiments of the present invention relates to the complex or the pharmaceutical composition of the present invention, for use in positron emission tomography (PET) imaging. One or more embodiments of the present invention relates to the complex or the pharmaceutical composition of the present invention, for use in single-photon emission computerized tomography (SPECT) imaging.

Positron emission tomography (PET) is a functional imaging technique that uses radioactive substances known as radiotracers to visualize and measure changes in metabolic processes, and in other physiological activities including blood flow, regional chemical composition, and absorption. Different tracers are used for various imaging purposes, depending on the target process within the body. PET is a common imaging technique, a medical scintillography technique used in nuclear medicine. A radiopharmaceutical—a radionuclide attached to a drug—is injected into the body as a tracer. The tracers of the present invention are the complexes and pharmaceutical compositions of the present invention where p-SCN-Bn-DOTA-PSMA is the compound or drug and the complex is the radiopharmaceutical.

Gamma rays are emitted and detected by gamma cameras to form a three-dimensional image, in a similar way that an X-ray image is captured. PET scanners can incorporate a CT scanner and are known as PET-CT scanners. PET scan images can be reconstructed using a CT scan performed using one scanner during the same session. Thus, in one or more embodiments of the present invention, the complexes and pharmaceutical compositions of the present invention are use in PET or PET-CT imaging.

Single-photon emission computed tomography (SPECT, or less commonly, SPET) is a nuclear medicine tomographic imaging technique using gamma rays. It is very similar to conventional nuclear medicine planar imaging using a gamma camera (that is, scintigraphy), but is able to provide true 3D information. This information is typically presented as cross-sectional slices through the patient but can be freely reformatted or manipulated as required. The technique needs delivery of a gamma-emitting radioisotope (a radionuclide of the present invention) into the patient, normally through injection into the bloodstream. Most of the time, though, a marker radionuclide is attached to a specific ligand to create a radioligand, whose properties bind it to certain types of tissues. This marriage allows the combination of ligand and radiopharmaceutical to be carried and bound to a place of interest in the body, where the ligand concentration is seen by a gamma camera. The radioligand of the present invention is the complex described above. Thus, in one or more embodiments of the present invention, the complexes and pharmaceutical compositions of the present invention are use in SPECT imaging.

In one embodiment, said imaging is for providing diagnosis, staging, and monitoring treatment of cancers.

In one embodiment, said imaging is for providing monitoring of cancers. Such monitoring does not involve a medical doctor and is essentially data from the imaging procedure. Over time these data can monitor changes in the cancer, such as progression or regression.

Thus, one or more aspect(s) of the present disclosure relates to compounds, complexes and pharmaceutical compositions according to the present disclosure, for use in in imaging.

The position emitting compounds, complexes and pharmaceutical compositions according to the present disclosure are usually prepared just prior to the imaging due to the relatively short half-life of the positron emitting nuclides.

The compounds, complexes and pharmaceutical compositions according to the present disclosure, usually comprises of a targeting molecule conjugated to a compound that is enriched in a positron emitting isotope.

Furthermore, compounds, complexes and pharmaceutical compositions according to the present disclosure may be enriched in ¹¹C, ¹³N, ¹⁵O or ¹⁸F.

### Kits

For kits, the compositions of the present invention should be made physiologically suitable for injections either at a centralized production site or be made up by a kit system of typically 2-4 vials whereby being physiologically suitable for injection after combination of the kit vials.

Thus, an aspect of the present invention relates to a kit comprising a first vial comprising a pharmaceutical composition according to the present invention, and a second vial comprising a neutralizing solution to adjust pH and/or isotonicity of the radiopharmaceutical composition prior to administration to a patient.

For, e.g., a monoclonal antibody, it is usually advisable be keep the self-dose of the alpha particle producing radiopharmaceutical solution below 0.5 kGy to avoid reduced binding properties due to radiolysis. Thus, a kit system whereby the compound of the present invention is added to a composition comprising the radionuclide a few hours to 10 minutes before injection is advised for concentrated solutions intended for remote shipping, depending of the radiolytic resistance of the radioligand that is generated.

One or more embodiments of the present invention relates to a kit comprising: a first vial comprising a pharmaceutical composition of the present invention, and a second vial comprising a neutralizing composition to adjust pH and/or isotonicity of the radiopharmaceutical composition prior to administration to a patient.

One or more embodiments of the present invention relates to a kit comprising: a first vial comprising a pharmaceutical composition comprising ²²⁷Th, ¹⁷⁷Lu, ²¹²Pb, ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb; and a second vial comprising p-SCN-Bn-DOTA-PSMA. The radionuclides can also be selected from the group consisting of ¹¹¹In, ²⁰³Pb, ⁸⁶Y, ⁹⁰Y, ⁸⁶Y, ⁸⁹Zr ⁶⁸Ga ⁶⁴Cu, ⁶⁷Cu, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb.

The kits can optionally comprise instructions for use.

### Medical Applications

An aspect of the present invention relates to the pharmaceutical composition according to the present invention for use as a medicament.

In one embodiment of the present invention is the disease cancer.

An aspect of the present invention relates to the pharmaceutical composition according to the present invention for use in the treatment of soft tissue and/or skeletal disease. The treatment is focused on PSMA-expressing disease including soft tissue- and skeletal disease.

In one embodiment of the present invention is the skeletal disease selected from the group consisting of soft tissue and or skeletal metastases from cancers to the breast, prostate, kidneys, lung, bone, or multiple myeloma.

In one embodiment of the present invention is the cancer prostate cancer. The cancer can also be breast cancer. The cancer can be kidney cancer. The cancer can also be lung cancer. The cancer can also be bone cancer. The cancer can also be multiple myeloma. The cancer can be metastases from these types of cancer.

Thus, the complexes and the solutions of the present invention can be used in the treatment of metastatic prostate cancer.

In one embodiment of the present invention is the solution administered at a dose in the range 50 kBq-500 MBq per kg of bodyweight, such as 50 kBq-100 MBq per kg of bodyweight.

An aspect of the present invention relates to a method of treatment of malignant or non-malignant disease by administration of a pharmaceutical composition according to the present invention to an individual in need thereof.

Another aspect of the present invention relates to a method of amelioration of malignant or non-malignant disease by administration of a pharmaceutical composition according to the present invention to an individual in need thereof.

Yet another aspect of the present invention relates to a method of inhibition of malignant or non malignant disease by administration of a pharmaceutical composition according to the present invention to an individual in need thereof.

### Methods for Preparation

An aspect of the present invention relates to a method for providing a pharmaceutical composition according to the present invention, the method comprising providing a first composition comprising the radionuclide; providing a second composition comprising a complexing agent (i.e. p-SCN-Bn-DOTA-PSMA), wherein the complexing agent is capable of complexing a the radionuclide, and mixing the first composition and the second composition, thereby providing a pharmaceutical composition according to the present invention.

### Activity Level

If the complexes of the present invention are used alone, the activity level would typically be between 1 MBq and 50 GBq per patient, more typically 10 MBq-20 GBq per patient. For alpha emitters typically in the lower end and for beta emitters typically in the higher end of the level interval. Also noteworthy is that the dosing can be given as single dose or as a repetitive dose of typically 2-10 repetitions. Thus, for alpha emitters the dose range is typically 5 MBq-500 MBq, For beta emitters the dose range is typically 1 GBq-50 GBq. For diagnostic use, the dose range is typically 100 MBq-10 GBq.

The dosing can also be between 0.1 MBq and 50 GBq per patient, more typically between 1 MBq and 20 GBq per patient.

The following figures and examples are provided below to illustrate the present invention. They are intended to be illustrative and are not to be construed as limiting in any way.

### EXAMPLES

### Example 1—Radiolabelling of p-SCN-Bn-DOTA-PSMA with ⁶⁸Ga

10 nmol of p-SCN-Bn-DOTA-PSMA were pre-heated for 5 min at 90° C.; after which 280 μl of ⁶⁸Ga from its stock solution and 5 μl of NH₄OAc (=50 μg/ml) were added. 5M NH₄OAc was used to adjust pH around 5-6. Activity of the solution was measured in a Capintec dose calibrator. The solution was then incubated at 90° C.; and 650 rpm and RCP was measured after 15 min utilizing Instant Thin Layer Chromatography (Model #150-772, Biodex Medical System Inc, Shirley, NY) and a Cobra gamma counter with a window between 50 and 2000 keV.

Radioligand binding at antigen excess using was evaluated by a one-point binding assay (RCP-corrected). The PSMA-expressing prostate cancer cell line C4-2 (ATCC CRL3314, Manassas, Virginia) 38 was grown as monolayer in RPMI 1640 medium (Sigma-Aldrich Norway AS, Oslo, Norway) supplemented with 10% heat inactivated fetal bovine serum (FBS, GE Healthcare Life Sciences, Chicago, Illinois), 100 units/mL penicillin and 100 μg/mL streptomycin (Sigma-Aldrich) at 37° C. in a humid atmosphere with 95% air and 5% CO₂. Cell binding of the radiolabelled ligands was verified by a similar cell binding assay. In this assay, 10 to 12×10⁶ cells were incubated with 1.5 to 6 nM of radioligand. The cell binding fraction (% cell bound activity of added activity) was estimated by subtracting nonspecific cell bound activity from total cell bound activity.

Results are presented in Table 1.

This example shows that p-SCN-Bn-DOTA-PSMA could be labeled with ⁶⁸Ga with a relevant yield and good cell binding ability of the radioligand.

TABLE 1
Main results after radiolabeling
of p-SCN-Bn-DOTA-PSMA with ⁶⁸Ga

| | |
|---|---|
| Specific Activity (MBq/nmol) | 0.3 |
| RCP (%) | 68.8 |
| % Cell binding | 65.6 |

### Example 2. Lead-212 Labeling of p-SCN-Bn-DOTA-PSMA as an Indicator for Preparation of ²⁰³Pb-Labeled p-SCN-Bn-DOTA-PSMA

25 nmol of p-SCN-Bn-DOTA-PSMA were pre-heated for 5 min at 37° C. after which 100 μl of ²¹²Pb from its stock solution (0.1 M HCl) prepared as describe (Li R G et al., 2023) and 10 μl of 5 M NH₄OAc were added. The 5 M NH₄OAc was used to adjust pH around 5-6. Activity of the solution was measured in a Capintec dose calibrator. The solution was then incubated at 37° C. and 650 rpm (Thermomixer Comfort, Eppendorf, Germany) and RCP was measured after 15 min utilizing Instant Thin Layer Chromatography (ITLC) (Model #150-772, Biodex Medical System Inc, Shirley, NY) and a Cobra gamma counter with a window between 50 and 2000 keV.

Radioligand binding at antigen excess was evaluated using a one point binding assay using the PSMA-expressing C4-2 cell line (ATCC CRL3314, Manassas, Virginia) which was grown as monolayer in RPMI 1640 medium (Sigma-Aldrich Norway AS, Oslo, Norway) supplemented with 10% heat inactivated fetal bovine serum (FBS, GE Healthcare Life Sciences, Chicago, Illinois), 100 units/mL penicillin and 100 μg/mL streptomycin (Sigma-Aldrich) at 37° C. in a humid atmosphere with 95% air and 5% CO2. Cell binding of the radiolabelled ligand was verified by cell binding assay. In this assay, 10 to 12×10⁶ cells were incubated with 1.5 to 6 nM of radioligand. Nonspecific binding was measured on cells pre-incubated with excess amounts of unlabeled ligand before addition of radioligand. The cell binding fraction (% cell bound activity of added activity) was estimated by subtracting nonspecific cell bound activity from total cell bound activity.

Results: Radiochemical purity of the radiolabelled product from two batches was determined by ITLC to be 94.5-98.7%. The cell binding fraction was found to be 47.3-55.0%.

In conclusion: When ²¹²Pb was used as an indicator for ²⁰³Pb labelling the data indicated a high and relevant labelling yield and appropriate cell binding ability of the radioligand.

### Example 3. Biodistribution of ²¹²Pb-Labeled p-SCN-Bn-DOTA-PSMA to Mimic 203Pb-p-SCN-Bn-DOTA-PSMA Radioligand

For the biodistribution study, male Hsd:Athymic Nude-Foxn1nu mice bred at the Department of Comparative Medicine at the Norwegian Radium Hospital (Oslo University Hospital, Oslo, Norway) were used.

Mice were inoculated subcutaneously in both flanks with 10×10⁶ C4-2 cells in supplement-free RPMI1640 medium mixed 1:1 with Matrigel Matrix (Corning, NY, USA) in a total volume of 200 uL. The tumours were allowed to grow to reach a volume of 300-1500 mm³ for the biodistribution studies and tumour-bearing mice were randomised based on tumour size before radioligand injection.

The studies were approved by the Institutional Committee on Research Animal Care (Department of Comparative Medicine, Oslo University Hospital) and the Norwegian Food Safety Authority (Brumunddal, Norway). All procedures and experiments involving animals in this study were performed in accordance with the Interdisciplinary Principles and Guidelines for the Use of Animals in Research, Marketing and Education (New York Academy of Sciences, New York, USA) and the EU Directive 2010/63/EU for animal experiments as well as the ARRIVE guidelines. The animals were maintained under specific pathogen-free conditions with ad libitum access to food and water. Cages (1-5 mice per cage) were housed in a scantainer, which was maintained at constant temperature (24° C.) and humidity (60%). The mice were around 4-6 weeks in age and weighed 25-35 g at the start of the study. The mice (N=3 per time point) were injected intravenously in the tail vein with 86 kBq of ²¹²Pb-p-SCN-Bn-DOTA-PSMA in 100 ul of isotonic saline solution and animals were sacrificed by cervical dislocation at 1 and 4 hours after injection. The % of injected activity per gram of tissue was calculated by measuring weight of- and radioactivity in-tissue samples and to determine tumor to tissue ratios the % of injected activity per gram tumor was divided by the % injected activity per gram of tissue.

Results: In Table 2, the ratios of tumor to muscle and tumor to bone are presented. The data shows very good uptake ratio between tumors and muscle and tumor and bone, indicating that p-SCN-Bn-DOTA-PSMA labeled with radioisotopes of lead, e.g. ²⁰³Pb, as a radioligand has promising properties for use in diagnostic nuclear imaging using e.g. gamma camera scanning.

TABLE 2
Uptake ratios of ²¹²Pb-p-SCN-Bn-DOTA-PSMA.

| Tissues | |
|---|---|
| Tumor/muscle | Tumor/bone |

| | | | | |
|---|---|---|---|---|
| Time point | 1 h | 4 h | 1 h | 4 h |
| (hours after | | | | |
| injection) | | | | |
| Ratios | 55.3 | 183.3 | 14.6 | 26.6 |

In conclusion: p-SCN-Bn-DOTA-PSMA is a promising molecule for radioligand imaging with e.g. single-photon emission computerized tomography (SPECT) and/or positron emission tomography (PET) radionuclides for use in diagnostic imaging of patients with PSMA expressing tumors.

### REFERENCE

Ruth Gong Li, Vilde Yuli Stenberg and Roy Hartvig Larsen. An Experimental Generator for Production of High-Purity 212Pb for Use in Radiopharmaceuticals. Journal of Nuclear Medicine January 2023, 64(1) 173-176;

1. A complex comprising;
a) a compound according to of the formula: 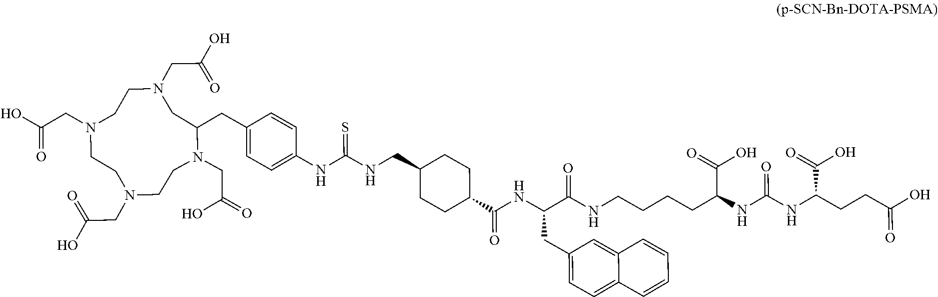 [SMILES: O=C(O)CC[C@H](NC(=O)N[C@@H](CCCCNC(=O)[C@H](Cc1ccc2ccccc2c1)NC(=O)[C@H]1CC[C@H](CNC(=S)Nc2ccc(CC3CN(CC(=O)O)CCN(CC(=O)O)CCN(CC(=O)O)CCN3CC(=O)O)cc2)CC1)C(=O)O)C(=O)O]
and
b) a radionuclide selected from the group consisting of ⁶⁸Ga, ²⁰³Pb, ¹¹¹In, ⁸⁶Y, ⁹⁰Y, ⁸⁶Y, ⁸⁹Zr, ⁶⁴Cu, ⁶⁷Cu, ¹⁷⁷Lu, ²²⁵Ac, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb.

2. The complex according to claim 1, wherein the radionuclide is selected from the group consisting of ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb.

3. The complex according to claim 1, wherein the radionuclide is ¹⁷⁷Lu.

4. The complex according to claim 2, wherein the radionuclide is ²²⁵Ac.

5. The complex according to claims 1-2, wherein the radionuclide is ¹¹¹In.

6. The complex according to claims 1-2, wherein the radionuclide is ⁸⁶Y.

7. The complex according to claims 1-2, wherein the radionuclide is ⁹⁰Y.

8. The complex according to claims 1-2, wherein the radionuclide is ⁸⁹Zr.

9. The complex according to claims 1-2, wherein the radionuclide is ⁶⁸Ga.

10. The complex according to claims 1-2, wherein the radionuclide is ⁶⁴Cu.

11. The complex according to claims 1-2, wherein the radionuclide is ¹⁶¹Tb.

12. The complex according to claims 1-2, wherein the radionuclide is ⁸⁶Y.

13. The complex according to claims 1-2, wherein the radionuclide is ⁶⁷Cu.

14. The complex according to claims 1-2, wherein the radionuclide is ⁴⁴Sc.

15. The complex according to claims 1-2, wherein the radionuclide is ⁴⁶Sc.

16. The complex according to claims 1-2, wherein the radionuclide is ⁴⁷Sc.

17. The complex according to claims 1-2, wherein the radionuclide is ⁴⁸Sc.

18. The complex according to claims 1-2, wherein the radionuclide is ¹⁵⁵Tb.

19. The complex according to claims 1-2, wherein the radionuclide is ¹⁴⁹Tb.

20. The complex according to claims 1-2, wherein the radionuclide is ²⁰³Pb.

21. The complex according to claims 1-2, wherein the radionuclide is ¹⁶¹Tb.

22. The complex according to claims 1-2, wherein the radionuclide is ⁴³Sc.

23. A pharmaceutical composition comprising the complex according to any one of claims 1-22, and a diluent, carrier, surfactant, and/or excipient.

24. The pharmaceutical composition according to claim 23, wherein pharmaceutical composition is dosaged with a radioactivity of 100 kBq to 50 GBq per dose.

25. The pharmaceutical composition according to claim 24, for use as a medicament.

26. The pharmaceutical composition according to claim 25, for use in the treatment of PSMA-expressing disease including soft tissue- and skeletal disease.

27. The pharmaceutical composition for use according to any one of claims 25-26, wherein the medicament is for use in the treatment of a disease selected from the group consisting of skeletal metastases from cancers to the breast, prostate, kidneys, lung, bone, or multiple myeloma.

28. The pharmaceutical composition for use according to any of claims 23-27, wherein the composition is administered at a dose in the range 50-150 kBq per kg of bodyweight.

29. The complex according to any one of claims 1-20 or the pharmaceutical composition according to any of claims 23-28, for use in imaging.

30. The complex according to any one of claims 1-22 or the pharmaceutical composition according to any of claims 23-28, for use in positron emission tomography (PET) imaging or single-photon emission computerized tomography (SPECT) imaging.

31. The complex according to any one of claims 1-22 or the pharmaceutical composition according to any of claims 23-28, for use according to any one of claims 29-30, wherein the imaging is for providing diagnosis, staging, and/or monitoring treatment of cancer.

32. A method of treatment of malignant or non-malignant disease by administration of a pharmaceutical composition according to any one of claims 23-28 to an individual in need thereof.

33. A method of amelioration of malignant or non-malignant disease by administration of a pharmaceutical composition according to any one of claims 23-28 to an individual in need thereof.

34. A method of inhibition of malignant or non-malignant disease by administration of a pharmaceutical composition according to any one of claims 23-28 to an individual in need thereof.

35. A kit comprising:
a first vial comprising a pharmaceutical composition according to any one of claims 22-28, and
a second vial comprising a neutralizing composition to adjust pH and/or isotonicity of the radiopharmaceutical composition prior to administration to a patient.

36. A kit comprising:
a first vial comprising a pharmaceutical composition comprising a radionuclide selected from the group consisting of ¹¹¹In, ²⁰³Pb, ⁸⁶Y, ⁹⁰Y, ⁸⁶Y, ⁸⁹Zr, ⁶⁸Ga, ⁶⁴Cu, ⁶⁷Cu, ⁴³Sc, ⁴⁴Sc, ⁴⁶Sc, ⁴⁷Sc, ⁴⁸Sc, ¹⁵⁵Tb, ¹⁴⁹Tb, and ¹⁶¹Tb;
a second vial comprising p-SCN-Bn-DOTA-PSMA.
